(19) United States
(12) Patent Application Publication
(10) Pub. No.: US 2014/0087408 A1
(43) Pub. Date: Mar. 27, 2014

(54) INFORMATION ACQUISITION METHOD

(71) Applicants: Yohei Murayama, Kawasaki-shi (JP); Hiroyuki Hashimoto, Yokohama-shi (JP); Manabu Komatsu, Kawasaki-shi (JP)

(72) Inventors: Yohei Murayama, Kawasaki-shi (JP); Hiroyuki Hashimoto, Yokohama-shi (JP); Manabu Komatsu, Kawasaki-shi (JP)

(73) Assignee: CANON KABUSHIKI KAISHA, Tokyo (JP)

(21) Appl. No.: 13/995,645

(22) PCT Filed: Nov. 28, 2011
(86) PCT No.: PCT/JP2011/078001

§ 371 Date: Nov. 25, 2013

(30) Foreign Application Priority Data
Dec. 20, 2010 (JP) 2010-283780

(51) Int. Cl. G01N 33/574

(52) U.S. Cl. CPC G01N 33/57415
USPC 435/23

(57) ABSTRACT

The present invention relates to an information acquisition method of acquiring information of distribution of a protein or peptide in a sample based on mass information obtained by mass spectrometry of the protein or peptide. The method includes mass spectrometry of a definite region of the sample after limited proteolysis of the protein or peptide and acquisition of information relating to distribution using an ion peak that has a two-dimensional intensity distribution having a Pearson product-moment correlation coefficient of 0.5 to more and 1.0 or less in the definite region against the two-dimensional intensity distribution of the parent ion of the protein or the peptide subjected to the limited proteolysis and has a peak intensity ratio of larger than 1.0 against the peak intensity of the integrated spectrum of the parent ion in the definite region, wherein the m/z of the ion peak is greater than 500.

### TECHNICAL FIELD

The present invention relates to an information acquisition method of acquiring information relating to distribution of a protein in body tissue based on mass information obtained by mass spectrometry of the protein.

### BACKGROUND ART

In the field of pathological examination, a technology of investigating expression of a specific antigen protein by immunostaining and making a definite diagnosis in the light of the result has been widely being used. In judgment of breast cancer, immunostaining is employed in detection of ER (estrogen receptor that is expressed in hormone-dependent tumor) as an indicator for determining hormone therapy and HER2 (membrane protein that is observed in rapidly progressive malignant cancer) as an indicator for determining Herceptin administration.

Recently, mass imaging by mass spectrometry has been developed as an analysis method for visualizing a protein at a cellular level. The mass imaging is a method of visualizing the two-dimensional distribution of a target material in a sample by mass spectrometry of each fragmented region of an arbitrary region of the sample and formation of an image using the ion peaks contained in the resulting mass spectra. The present inventors have proposed (PTL 1) time-of-flight secondary ion mass spectrometry (TOF-SIMS) as a method of measuring two-dimensional distribution of a peptide fragment (hereinafter also referred to as “digestion fragment”) produced by limited proteolysis (herein after also referred to as “digestive proteolysis”) of the surface of a body tissue section with a digestive enzyme. Herein, the term “limited proteolysis” means that a peptide bond between a specific amino acid residue and its adjacent amino acid residue in a protein is selectively cleaved to produce a digestion fragment smaller than the protein.

Furthermore, as mass imaging for visualizing an expressed protein, application of matrix-assisted laser desorption ionization (MALDI) has been developed. The MALDI is a method for ionizing a sample through crystallization by mixing the sample into a matrix and irradiation of the crystal with laser. As mass imaging using the MALDI, it has been disclosed visualization of an expression status of HER2 by obtaining an image of a lesion tissue section expressing HER2 by selecting an ion peak based on the immunostaining image of HER2 (NPL 1).

### CITATION LIST

### Patent Literature

PTL 1 Japanese Patent Laid-Open No. 2006-010658

### Non Patent Literature

NPL 1 S. Rauser, et al., Journal of Proteome Research, 2010, 9, 1854-1863

### SUMMARY OF INVENTION

### Technical Problem

However, in the method forming a two-dimensional distribution image of a protein of a body tissue section by performing TOF-SIMS after digestive proteolysis of the protein and using the signal of a theoretical digestion fragment, it is difficult to obtain the image at a high contrast.

In the method using the MALDI and immunostaining described in NTL 1, an image corresponding to HER2 on a body tissue section is obtained. However, this method uses an immunostaining image, which takes a long time for visualization.

### Solution to Problem

The present invention provides an information acquisition method of acquiring information relating to distribution of a protein or a peptide in a sample based on mass information obtained by mass spectrometry of the protein or the peptide. The method includes mass spectrometry of a definite region of the sample after limited proteolysis of the protein or peptide and acquisition of information relating to distribution using an ion peak that has a two-dimensional intensity distribution having a Pearson product-moment correlation coefficient of 0.5 to more and 1.0 or less in the definite region against the two-dimensional intensity distribution of the parent ion of the protein or the peptide subjected to the limited proteolysis and has a peak intensity ratio of larger than 1.0 against the peak intensity of the integrated spectrum of the parent ion in the definite region, wherein the m/z of the ion peak is greater than 500.

### Advantageous Effects of Invention

According to the present invention, an image obtained by visualization of a protein in body tissue by mass imaging can be improved in contrast.

### BRIEF DESCRIPTION OF DRAWINGS

FIG. 1 schematically shows the visualization process of mass information according to the present invention.

FIG. 2 is an image visualized by using a parent ion peak of a digestion fragment at m/z 1438.3 in Comparative Example 1.

FIG. 3 is an image visualized by using an ion peak at m/z 719.7 in Example 1.

FIG. 4 is an image visualized by using an ion peak at m/z 1267.7 in Example 1.

FIG. 5 is an image visualized by using an ion peak at m/z 1298.0 in Example 1.

FIG. 6 is an immunostaining image of an adjacent section.

FIG. 7 is a graph showing a correlation between expression levels and TOF-SIMS average signal intensities (standard values) at m/z 719.7.

### DESCRIPTION OF EMBODIMENTS

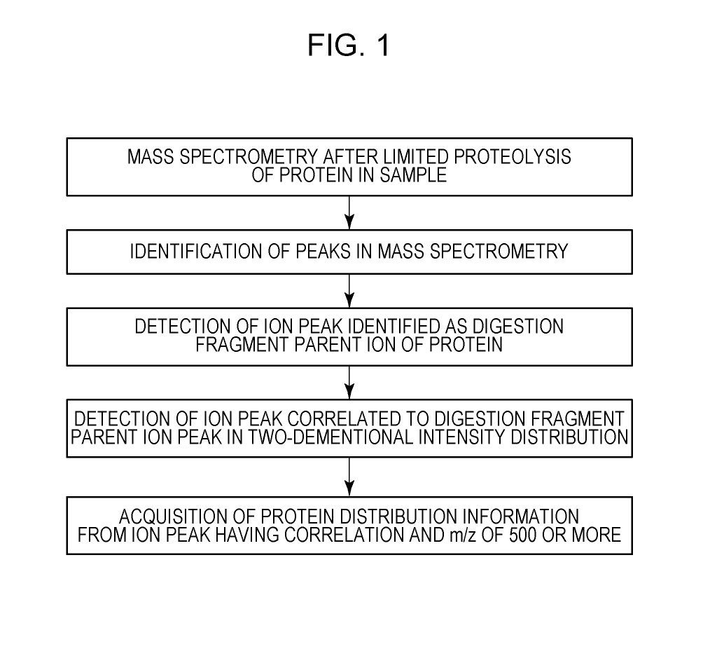

The information acquisition method of the present invention can acquire information of distribution of a protein by the process shown in FIG. 1.

In the first step, after limited proteolysis of a protein in a sample, a definite region of the sample is subjected to mass spectrometry (step of mass spectrometry). In the second step, the mass information of the mass spectrum obtained by the mass spectrometry is compared with an amino acid sequence database of proteins to identify amino acid sequences and to be assigned to the parent ions of specific digestion fragments of the peaks (step of assignment of peaks obtained by mass spectrometry). In the third step, from the identified and assigned peaks, a peak assigned as the parent ion of a digestion fragment of the protein is detected (step of detecting parent ion peak of digestion fragment). In the fourth step, an ion peak correlated to the parent ion peak of the detected digestion fragment in two-dimensional intensity distribution is detected (step of detecting ion peak correlated in two-dimensional intensity distribution). In the fifth step, information of protein distribution is acquired using the ion peak obtained in the fourth step, wherein the ion peak has an m/z greater than 500 and is correlated to the parent ion of the digestion fragment of the protein in the two-dimensional intensity distribution (step of acquiring information of protein distribution).

In the case of acquiring information of protein distribution of a plurality of samples, distributions of proteins on and after the second sample can be each visualized by performing the fifth step using the information of an ion peak obtained in the fourth step in the first sample without performing the second to fourth steps.

Each step will be described in detail below.

### Step of Mass Spectrometry

In the step of mass spectrometry, a protein in a sample such as body tissue is subjected to limited proteolysis, and a definite region of the sample is subjected to mass spectrometry.

The material (hereinafter referred to as “digestion material”) used in limited proteolysis (digestive proteolysis) of a protein is not particularly limited. Examples of the digestion material are roughly classified into (I) digestive enzymes and (II) chemical materials other than the digestive enzymes. Typical examples of the digestive enzymes (I) include trypsin, chymotrypsin, Lys-C, and Asp-N. Typical examples of the chemical materials (II) other than the digestive enzymes include cyanogen bromide (CNBr), 3-methyl-3-bromo-2-[(2-nitrophenyl)thio]-3H-indole (BNPS-skatole), 2-nitro-5-thiocyanobenzoate (NTCB), hydroxyalanine, formic acid, dimethyl sulfoxide-hydrochloric acid-hydrogen bromide (DMSO-HCl-HBr), and N-bromosuccinimide (NBS).

In the present invention, one or more digestion materials are used. The position of a peptide bond that is cleaved depends on the type of the digestion material. For example, in the case of using trypsin, a peptide bond on the carboxyl group side of lysine (K) and/or arginine (R) is cleaved. In the case of using NTCB, a peptide bond on the amino group side of cysteine (C) is cleaved.

In the digestive proteolysis of a protein using the digestion material, a solution is used by adjusting the conditions, such as pH, to be suitable for the digestion reaction. The solution containing the digestion material is dropwise added to a protein using, for example, a micropipette, an ink-jet, or a sprayer. Alternatively, the digestion material can be added to a protein by leaving the protein in an environment filled with vapor of the solution containing the digestion material. In both addition methods, the digestion reaction proceeds by leaving the protein together with the digestion material in an environment of a specific temperature or humidity for about several hours to several tens hours. For example, in the case of using trypsin as the digestion material, since the reaction well proceeds at 37 to 38° C., the protein is left in an environment of a temperature of 37 to 38° C.

In addition, pretreatment for allowing efficient progress of the digestion reaction may be performed before the digestion reaction. For example, in the case of using trypsin as the digestion material, reductive alkylation treatment by adding dithiothreitol (DTT) and iodoacetamide to the protein can be performed before the addition of trypsin.

The sample that is visualized by the present invention contains a protein or a peptide. The protein or the peptide is decomposed into digestion fragments having smaller molecular weights by the limited proteolysis treatment.

Then, the protein or the peptide in the sample after the limited proteolysis is subjected to mass spectrometry to obtain a mass spectrum.

The mass spectrometry in this step is not particularly limited. The apparatus for the mass spectrometry has a sample feeding portion for performing ionization of a sample and an analysis portion for performing analysis of the ionized sample. The methods for the mass spectrometry are classified according to the system of the analysis portion.

The ionization in the sample feeding portion can be performed by the following method: a method using primary ions, matrix-assisted laser desorption ionization (MALDI), desorption electrospray ionization (DESI), or fast atom bombardment (FAB).

The DESI is a method in which charged droplets are sprayed onto the surface of a sample to desorb ions from the sample surface.

The FAB is a method in which a sample is mixed into a matrix and is bombarded with neutral atoms at a high speed for ionization.

The system of the analysis portion can be any of the followings:

(a) Quadrupole type,
(b) Magnetic deflection type,
(c) Fourier transform ion cyclotron resonance type,
(d) Ion trap type,
(e) Time-of-flight (TOF) type, and
(f) tandem type.

Here, the quadrupole type (a) is an analysis method in which ions are allowed to pass through among four electrodes, the electrodes are applied with a high-frequency voltage to cause perturbation of a sample in such a manner that only target ions pass through the electrodes. The magnetic deflection type (b) is an analysis method in which a change in the flight path of ions due to a Lorentz force when the ions pass through a magnetic field is utilized. The Fourier transform ion cyclotron resonance type (c) is an analysis method in which ions are introduced into a cell applied with an electrostatic field and a magnetostatic field, a high-frequency voltage for exciting ion motion is applied to the cell to detect the orbiting period of the ions, and the mass is calculated from the cyclotron conditions. The ion trap type (d) is an analysis method in which ions are held in a trap chamber having an electrode, and ions are selectively released by changing the potential to cause separation. The tandem type (f) is a method of a combination of the above-described analysis methods.

In mass spectrometry employing any of the above-mentioned methods and systems in the sample feeding portion and the analysis portion, a high molecular protein or peptide in a sample is decomposed into low molecular compounds by limited proteolysis of the protein or peptide to allow high-sensitive measurement of a body tissue surface.

However, in some of mass spectrometry methods employed for mass imaging, treatment for ionization of a sample is indispensably required. For example, in the case of performing ionization by MALDI, a material absorbing laser light energy, called a matrix agent, is added to a sample after the limited proteolysis. Typical examples of the matrix agent are organic acid matrix molecules such as nitroanthracene (9NA), 2,5-dihydroxybenzoic acid (DHB), sinapinic acid (SA), and α-cyano-hydroxy-cinnamic acid (CHCA). In addition, a fine powder of a metal such as cobalt or a matrix of a liquid such as glycerol can be used. Furthermore, urea or lipid can be used as an energy absorber for infrared laser. In the case of employing MALDI in the present invention, one or more types of matrix agents are used. In the present invention, the type of the matrix agent is not limited.

In the case of using primary ions in ionization and employing time-of-flight type secondary ion mass spectrometry (TOF-SIMS) (e) in the analysis portion, the detection sensitivity can be improved by addition of a material enhancing ionization (hereinafter referred to as “ionization enhancing material”), which has been proposed by the present inventors (U.S. Pat. Nos. 7,446,309 and 7,701,138). As the ionization enhancing material, a material containing at least one of metal elements such as Ag and Au and alkali metal elements such as Na and K can be used, or an aqueous solution containing an acid such as trifluoroacetic acid and having a pH of 6 or less can be used.

In the present invention, primary ions can be used in ionization, and time-of-flight type secondary ion mass spectrometry (TOF-SIMS) (e) can be used in the analysis portion. The TOF-SIMS is a mass spectrometry method that allows highly sensitive measurement using a trace amount of a sample. By ionizing the sample through irradiation of the surface of the sample with primary ions in a pulsed manner, the sample is prevented from being damaged, and distribution information of the target material can be obtained with high precision and accuracy.

In the TOF-SIMS, the ionization of a sample is performed by irradiation with primary ions. As the primary ion species, cluster ions such as Au³⁺ and Bi³⁺, as well as general liquid metal ions such as Ga⁺, can be used from the viewpoints of, for example, ionization efficiency and mass resolution. The use of Bi ions allows significantly sensitive analysis and is therefore advantageous. Not only Bi ions but also polyatomic ions of bismuth, Bi₂ ions or Bi₃ ions, can be used, and the sensitivity is increased in this order in many cases. The same effect can be expected in polyatomic ions of gold.

In the TOF-SIMS, secondary ions are generated on the surface of a target material by incidence of primary ions. During the analysis by the TOF-SIMS, an electric field of several kilovolts is applied between the target material and a time-of-flight secondary ion mass spectrometer, and the secondary ions are incorporated into a detector by this electric field.

In the case of imaging by the TOF-SIMS, conditions such as mass resolution, analysis area, and measurement conditions, e.g., the primary ion pulse frequency, the primary ion beam energy, and primary ion pulse width, are closely involved in imaging ability. Accordingly, optimum analysis conditions are not unambiguously determined simply. Typical conditions are, for example, a primary ion pulse frequency of 1 to 50 kHz, a primary ion beam energy of 12 to 25 keV, and a primary ion beam pulse width of 0.5 to 10 ns. The mass spectrum of the measurement target can be obtained by scanning a pixel surface of 64 to 512 pixels square in a measurement region of a 10 to 500 μm square with the primary ion beams 16 to 512 times repeatedly. In the case of a broad measurement region (larger than 500 μm square), a mass spectrum of a broad region can be obtained using a raster-scan mode for scanning by operating the stage.

### Step of Assignment of Peaks Obtained by Mass Spectrometry

In the step of assignment of peaks obtained by mass spectrometry, peaks are identified by comparing the mass information (m/z values) of ion peaks of the mass spectrum obtained in the step of mass spectrometry with an amino acid sequence database of proteins. Then, identification of an amino acid sequence and assignment to the parent ion of a specific digestion fragment are performed.

Here, the term “parent ion” refers to an ion (M^{+•}) of a molecule (M) ionized by desorption of an electron or addition of a specific ion and in a fragmentation-free state. Typical examples of the ion generated by addition of an ion include hydrogen ion adducts ([M+H]⁺), sodium ion adducts ([M+Na]⁺), potassium ion adducts ([M+K]⁺), and calcium ion adducts ([M+Ca]⁺ or [M+Ca]²⁺). Examples of the parent ion also include adducts of metal ions, solvent-derived ions, and ions derived from the matrix on the periphery of a molecule.

More specifically, the identification of an amino acid sequence and the assignment to the parent ion of a specific digestion fragment are performed by any of the following procedures:

(I) collation of the mass information obtained by mass imaging with an amino acid sequence database of proteins, or
(II) collation of the mass information obtained by mass imaging with the amino acid sequence information that has been obtained by collation of the mass information obtained by another mass spectrometry with an amino acid sequence database of proteins.

The identification of an amino acid sequence by the collation of mass information with a protein database in the above (I) and (II) is roughly classified into a case of that the mass information is of a parent ion only and a case of that the mass information is of a parent ion and its fragment ions resulting from decomposition by mass spectrometry of the parent ion. The former is called peptide mass finger printing (PMF), and the latter is called MS/MS ion search.

In any of the identification methods, the species of a protein contained in the measured body tissue can be specified, and identification of amino acid sequence information of the obtained parent ion and assignment as a parent ion of the digestion fragments generated from the protein can be simultaneously performed. However, in the MS/MS ion search, data relating to fragment ions (hereinafter referred to “MS/MS data”) is necessary, and the quantity of the data is larger than that in the PMF, resulting in complication. However, the MS/MS ion search can survey continuous amino acid sequence information and thereby can identify proteins with higher accuracy than the PMF.

The procedures (I) and (II) in this step can employ any of the identification methods. However, in the case of employing MS/MS ion search when MS/MS data is not obtained, the procedure (II) is performed. A case of performing the procedure (II) in MS/MS ion search will be described in detail below.

First, protein identification is separately performed by MS/MS ion search using another mass spectrometry method, and amino acid sequence information is acquired for the parent ion obtained by another mass spectrometry method. Then, the acquired amino acid sequence information is collated with the parent ion obtained by the mass spectrometry in the mass spectrometry step to perform identification of the amino acid sequence and assignment to the parent ion of a specific digestion fragment for the ion peak obtained by the mass spectrometry in the mass spectrometry step.

In the collation of the amino acid sequence information obtained by MS/MS ion search with the parent ion obtained by the mass spectrometry in the mass spectrometry step, m/z values of monoisotopic ion peaks of the both are compared. On this occasion, the mass accuracy of the mass imaging is adopted. That is, when the difference between the m/z of the monoisotopic ion peak obtained by the mass spectrometry in the mass spectrometry step and the theoretical m/z of the monoisotopic ion peak obtained from the amino acid sequence identified by the MS/MS ion search is within the margin of error determined based on the mass accuracy, the ion peak obtained by the mass spectrometry is judged to coincide with that of the amino acid sequence and is assigned to the parent ion of the specific digestion fragment. If the difference is larger than the margin of error, the ion peak is judged to be discordant.

In the collation, in addition to the comparison of m/z values of monoisotopic ion peaks, comparison of an actually measured spectrum and a theoretical spectrum for the isotopic distribution shapes of the parent ions may be performed as a judgment factor.

Typical examples of the mass spectrometry for obtaining MS/MS data include, but not limited to, LC-MS/MS. Typical examples of the amino acid sequence database of proteins include, but not limited to, NCBInr available from the National Center for Biotechnology Information and SwissProt available from the UniProt Consortium. Typical examples of the search engine for collating mass information with the protein database include, but not limited to, MASCOT available from Matrix Science Ltd. and SEQUEST available from Thermo Fisher Scientific Inc.

### Step of Detecting Parent Ion Peak of Digestion Fragment

In the step of detecting the parent ion peak of a digestion fragment, amino acid sequences are identified in the step of assignment of the peaks obtained by the mass spectrometry, and from the ion peaks assigned to the parent ion of a specific digestion fragment, at least one ion peak assigned as the parent ion of the digestion fragment generated from a target protein or peptide is detected.

The ion peak detected in this step may be a monoisotopic ion peak or another isotopic peak.

### Step of Detecting Ion Peak Correlated in Two-Dimensional Intensity Distribution

In the step of detecting an ion peak correlated in two-dimensional intensity distribution, detected is an ion peak of which two-dimensional intensity distribution correlates to the parent ion peak of the digestion fragment obtained in the step of detecting parent ion peak of digestion fragment. Specifically, the detection is performed by the following method.

First, the mass information of the parent ion peak of the digestion fragment generated from a protein in a sample, which has been obtained in the step of detecting parent ion peak of digestion fragment, is visualized. In this occasion, the mass information may be visualized using only one parent ion peak, or the mass information as the sum of a plurality of parent ion peaks may be visualized.

Then, the mass information of ion peaks other than the parent ion peak of the digestion fragment generated from the target protein or peptide in the sample, which has been obtained in the step of detecting parent ion peak of digestion fragment, is visualized. On this occasion, peaks that can be judged as isotropic ion peaks of the specific monoisotopic ion peak may be excluded or may be added to the monoisotopic ion peak, in the visualization of mass information.

Then, a correlation of a two-dimensional intensity between the visualized image of the parent ion peak of the digestion fragment and the visualized images of the peaks other than the parent ion peak of the digestion fragment is evaluated. The correlation can be evaluated using the correlation coefficients of the images. The correlation coefficient of an image is an index for evaluating correlation (similarity) between two different images as the variables. Such a correlation coefficient is also called cross-correlation coefficient. In the present invention, the coefficient is simply called correlation coefficient. In the correlation coefficient, there are some types such as Pearson product-moment correlation coefficient and correlation coefficient calculated by Fourier transform. In any of the methods, in two different images of X pixels horizontally and Y pixels vertically, the signal intensity f(x,y) at one pixel position (x,y) (herein, x=1 to X, and y=1 to Y) and the signal intensity g(x,y) at another pixel position (x,y) (herein, x=1 to X, and y=1 to Y) are used as the variables.

The correlation coefficient of the visualized image of each peak other than the parent ion of the digest fragment is calculated. The resulting coefficients are sorted in order of being close to 1, and one or more ion peaks ranked high are detected as the correlating ion peak or peaks.

The correlating ion peak means that when the correlation coefficient of the visualized image of the mass information of an ion peak is closer to 1, the two-dimensional intensity distribution of the peak is closer to that of the parent ion peak of the digested fragment. The correlating ion peak can be one that has been ranked high in the sorting.

The correlating ion peak can have a Pearson product-moment correlation coefficient of 0.5 or more and 1.0 or less, such as 0.6 or more and 1.0 or less. An ion peak having such a correlation coefficient can be presumed as a peak of an intermediate product or a by-product in the production of the protein.

### Step of Acquiring of Information of Protein Distribution

In the step of acquiring of information of protein distribution, information of protein distribution is acquired using ion peaks at m/z 500 or more, in the ion peaks having correlation detected in the step of detecting ion peaks correlated in the two-dimensional intensity distribution. Herein, in the acquisition of distribution information of a protein, visualization and imaging of the protein distribution information are included. When the value of m/z is lower than 500, in the ion peaks correlated in the two-dimensional intensity distribution, for example, ion peak fragments that are unrelated to the parent ion and ion peaks derived from lipids are included. Therefore, when the value of m/z is lower than 500, it is difficult to acquire mass information of only a protein related to the protein.

The peak intensity ratio of the ion peak used in this step to the peak intensity of integrated spectrum in a definite region in which the mass spectrometry of the parent ion of a protein or peptide subjected to limited proteolysis is performed is larger than 1.0, preferably larger than 2.0, more preferably larger than 3.0, and most preferably larger than 10.0. Here, the integrated spectrum of the definite region is a spectrum obtained by integrating spectra stored in all pixels in the measurement region.

When the digestion material is trypsin and the protein or peptide subjected to limited proteolysis is (the parent ion of) HER2 in the step of mass spectrometry, information of protein distribution can be acquired using the following ion peak. The ion peak to be used is one or more ion peaks of which mass-to-charge ratio (m/z) is selected from 719.7±0.5, 1267.7±0.5, and 1298.0±0.5. In particular, an ion peak of which mass-to-charge ratio is 719.7±0.5 can give an image of mass information at a high contrast and therefore can be advantageously used.

### EXAMPLES

The present invention will be more specifically described with reference to examples below. The following specific examples are one embodiment, and the present invention is not limited to such a specific embodiment.

### Comparative Example 1

In Comparative Example 1, a protein in body tissue was subjected to digestive proteolysis with reference to Japanese Patent Laid-Open No. 2006-010658. A parent ion peak of a digestion fragment of the protein subjected to limited proteolysis was detected by TOF-SIMS, and the protein was visualized using an ion peak having correlation with the parent ion in the two-dimensional intensity distribution.

In this comparative example, HER2 was used as the protein, and trypsin was used as the digestive enzyme in limited proteolysis.

The procedure of this comparative example will be described below.

In this comparative example, as a body tissue section for analysis, a commercially available paraffin fixed human breast cancer tissue section, purchased from US Biomax, Inc., overexpressing HER2 was used. The procedure of preparing a sample for analysis will be described below.

First, deparaffinization was performed by washing the body tissue section with xylene, ethanol, and pure water. Then, trypsin digestion treatment was performed. Trypsin, purchased from Sigma-Aldrich Corp., was dissolved in an aqueous solution of ammonium bicarbonate, purchased from Kishida Chemical Co., Ltd., at a concentration of 0.05 μg/μL (pH 8.5). Ten microliters of the resulting trypsin solution was dropwise added onto the tissue section using a micropipette, and then the tissue section was left in an environment of 38° C. for 3 hr for enhancing the digestion reaction. Then, an aqueous solution of 0.1% trifluoroacetic acid (TFA) serving as an ionization enhancing material was dropwise added onto the tissue section after the digestion treatment using a micropipette, and the tissue section was dried in a room temperature environment.

The surface of the thus prepared tissue section was subjected to TOF-SIMS. The TOF-SIMS was performed using a TOF-SIMS V instrument manufactured by ION TOF GmbH. The measurement conditions were as follows:

Primary ion: 25 kV Bi⁺, 1 pA (pulsed current value), and stage raster-scan mode,
Primary ion pulse frequency: 5 kHz (200 μs/shot),
Primary ion pulse width: about 1 ns,
Primary ion beam diameter: about 2 μm,
Measurement region: 4×4 mm,
Number of pixels of secondary ion image: 256×256,
Number of shots per pixel: 256 shots, and
Detected secondary ion: positive ion.

Then, a peak was identified by comparing the mass information (m/z value) of mass spectrum obtained by the mass spectrometry with an amino acid sequence database of proteins. Then, identification of an amino acid sequence and assignment to the parent ion of a specific digestion fragment of the peak were performed. The identification and the assignment were performed by collating (1) the mass data obtained by LC-MS/MS analysis of a solution after trypsin digestion of cultured cells (N87 cell line, manufactured by ATCC) overexpressing HER2 with (2) the amino acid sequence information of the parent ion peak identified by automated collation search with database of human-derived proteins and with (3) the TOF-SIMS ion peak. The LC-MS/MS measurement was performed using a Paradigm MS4 apparatus manufactured by Michrom BioResources, Inc. and an LTQ Orbitrap XL apparatus manufactured by Thermo Fisher Scientific Inc. As the protein database, an extract from SwissProt provided by the UniProt Consortium was used. As the search engine, SEQUEST available from Thermo Fisher Scientific Inc. was used.

The mass spectrum thus-obtained by the TOF-SIMS was subjected to identification of amino acid sequence and assignment to a specific digestion fragment parent ion. The results were that a peak having an m/z of 1438.3 was assigned as the parent ion peak of a trypsin digestion fragment peptide, AVTSANIQEFAGCK (amino acid sequence), of HER2.

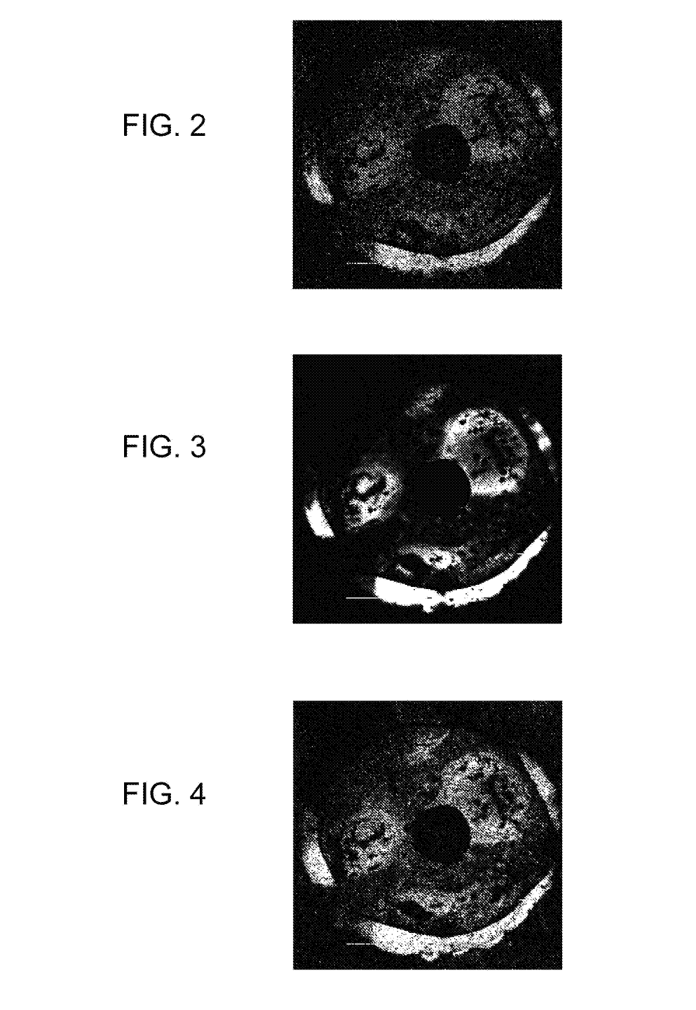

Then, an image of the two-dimensional distribution was formed using the parent ion (m/z 1438.3) of the HER2 digestion fragment. The result is shown in FIG. 2.

### Example 1

In this example, as in Comparative Example 1, HER2 was used as a sample, trypsin was used as the digestive enzyme for limited proteolysis, and TOF-SIMS was employed as the mass spectrometry.

In this example, as in Comparative Example 1, a commercially available paraffin fixed human breast cancer tissue section (manufactured by US Biomax, Inc.) overexpressing HER2 was used.

First, the paraffin fixed human breast cancer tissue section was subjected to treatment as in Comparative Example 1 and was subjected to TOF-SIMS. Identification and assignment of the obtained mass spectrum were performed. An image of the two-dimensional distribution was formed using the parent ion (m/z of 1438.3) of the HER2 digestion fragment.

Then, images of two-dimensional distribution were formed for peaks that were obtained by TOF-SIMS measurement and were not identified as the parent ion of the HER2 digestion fragment.

Correlation between the images visualized using the peaks that were not identified as the parent ion of the digestion fragment generated from HER2 and the image obtained using the parent ion peak (m/z 1438.3) of the digestion fragment generated from HER2 was evaluated using the Pearson product-moment correlation coefficient.

The results of determination of the correlation coefficients of the images were that images of m/z 719.7, 1267.7, and 1298.0 were showed high correlation to the image formed from the parent ion peak (m/z 1438.3) of the digestion fragment generated from HER2. The correlation coefficients of these images with the image formed from the parent ion peak of the digestion fragment generated from HER2 were 0.69, 0.57, and 0.57, respectively.

The ratios of the integrated spectrum intensity of the definite region of each of the peaks at m/z 719.7, 1267.7, and 1298.0 obtained above to the integrated spectrum intensity of the definite region of the parent ion peak (m/z 1438.4) of the digestion fragment generated from HER2 were determined. The results were 12.2, 3.2, and 2.1, respectively.

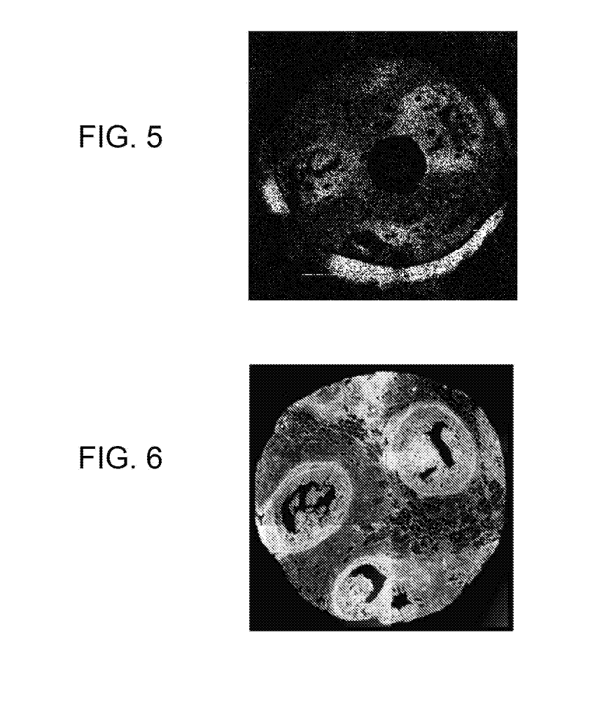

An image was formed from the above for each of the ion peaks (m/z 719.7, 1267.7, and 1298.0) having an m/z larger than 500 and having a Pearson product-moment correlation coefficient of 0.5 or more and 1.0 or less and an intensity ratio of larger than 1.0 in the integrated spectrum of the definite region against the two-dimensional intensity distribution of HER2. FIGS. 3, 4, and 5 are images visualized peaks at m/z 719.7, 1267.7, and 1298.0, respectively. FIG. 3 is the image with the highest contrast.

### Comparative Example 2

Images of two-dimensional distribution were formed using the ion peaks in Example 1 excepting the ion peak formed into an image in Example 1. The correlation coefficients in the following examples were lower than that of the image in Example 1.

The correlation coefficients of the images formed using the ion peaks at m/z 559.3 and 647.5 were 0.45 and 0.23, respectively. These ion peaks had m/z values close to that of the parent ion of the digestion fragment generated from HER2, but were not assigned to the parent ions of the digestion fragment in the step of identification and assignment. These ion peaks were conjectured as (1) ions that were not the parent ion of the digestion fragment generated from HER2, though the m/z values were close to that of the parent ion, or (2) ions included the parent ions of the digestion fragment generated from HER2, but the correlation coefficients thereof were decreased due to the present of other ions having m/z values close to that of the parent ion or the low detection sensitivity.

The correlation coefficients of images formed from the peaks at m/z 413.3, 643.5, 699.5, 1011.5, and 1228.8 were −0.15, 0.37, 0.42, 0.25, and 0.27, respectively. The m/z values of these ion peaks were close to that of the parent ion of digestion fragment generated from a house keeping protein. These peaks were presumed that the peak at m/z 413.3 was a protein related to albumin, the peak at m/z 643.5 was a protein related to actin, the peaks at m/z 699.5 and m/z 1011.5 were related to napsin, and the peak at m/z 1288.8 was related to tubulin. Consequently, it was conjectured that these ion peaks were parent ions of digestion fragments generated from proteins that were not related to HER2 and that the correlation coefficients were therefore small.

### Evaluation

In the comparison of the images (FIGS. 3 to 5) obtained in Example 1 with the image (FIG. 2) obtained in Comparative Example 1, the images obtained in Example 1 were obviously clearer than the image of Comparative Example 1.

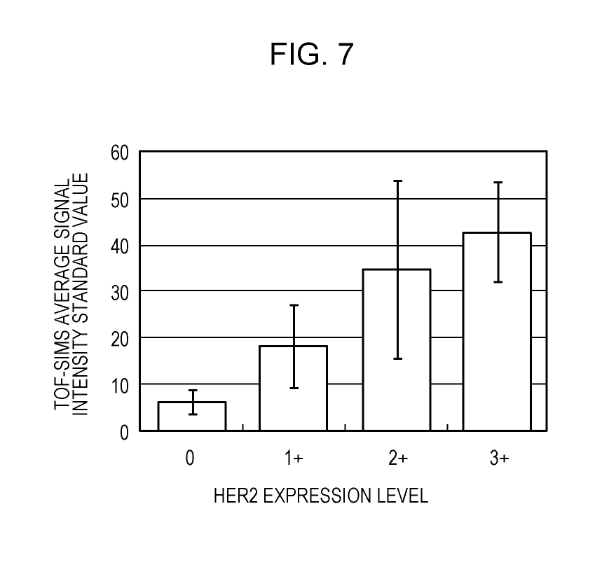

In the comparison of the images (FIGS. 2 and 3 to 5) obtained in Example 1 and Comparative Example 1 with the optical microscope image (FIG. 6) obtained by immunostaining of an adjacent section of the tissue section, the both well agreed. Images of a plurality of paraffin-fixed human breast cancer tissue sections expressing HER2 with different levels were formed as in Example 1. The average signal intensity of the images correlated well with the expression levels. FIG. 7 shows the results of evaluation of the image formed from the peak at m/z 719.7 for correlation. In FIG. 7, in order to correct the difference in ionization rate among samples, a standard value obtained by dividing the average signal intensity of ion peak at m/z 719.7 by the average signal intensity of ion peak at m/z 405.0, which can give a strong signal in every region of each sample, was used. The assignment of the peak at m/z 405.0 was not sufficiently performed, but it was presumed from the mass information that the peak was of the parent ion of a digestion fragment generated from a protein related to actin. It is believed based on the above that the image obtained in Example 1 is an image formed using the ion peak related to HER2 expression.

The above-described evaluation confirmed that in visualization of HER2 expressed in a tissue section, the images formed using an ion correlated to the parent ion of the HER2 digestion fragment as in Example 1 are clearer than the images formed using the parent ion of the HER2 digestion fragment as in Comparative Example 1.

In the above example, a commercially available paraffin-fixed human breast cancer tissue section overexpressing HER2 was subjected to digestion proteolysis with trypsin, followed by measurement by TOF-SIMS. But the present invention is not limited thereto.

For example, in the case of using another digestion material instead of trypsin, the cleavage position of a peptide bond constituting a protein varies, but the method described in this example can be also applied to such a case.

For example, in the case of using NTCB as the digestion material, typical examples of the parent ion of theoretical digestion fragment generated from HER2 include those of m/z 855.4 ([CKGPLPTD+CN]⁺), 1052.5 ([CLHFNHSGI+CN]⁺), and 1102.5 ([CAHYKDPPF+CN]⁺). On this occasion, as in Example 1, a clear image can be obtained by detecting an ion peak assigned as the parent ion of the theoretical digestion fragment, then detecting an ion peak of which ion two-dimensional intensity distribution correlates to the assigned ion peak serving as the standard, and forming an image using the correlated ion peak.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-283780, filed Dec. 20, 2010, which is hereby incorporated by reference herein in its entirety.

1. An information acquisition method of acquiring information relating to distribution of a protein or a peptide in a sample based on mass information obtained by mass spectrometry of the protein or the peptide in the sample, the method comprising:
mass spectrometry of a definite region of the sample after limited proteolysis of the protein or the peptide; and
acquisition of information relating to distribution using an ion peak,
wherein the ion peak has a two-dimensional intensity distribution having a Pearson product-moment correlation coefficient of 0.5 to more and 1.0 or less in the definite region against the two-dimensional intensity distribution of the parent ion of the protein or the peptide subjected to the limited proteolysis and has a peak intensity ratio of larger than 1.0 against the peak intensity of the integrated spectrum of the parent ion in the definite region,
wherein the m/z of the ion peak is greater than 500.

2. The information acquisition method according to claim 1, wherein the mass spectrometry is TOF-SIMS.

3. The information acquisition method according to claim 1, wherein the protein is HER2, the digestive enzyme for the limited proteolysis is trypsin, and the ion peak is composed of one or more peaks wherein the m/z of each ion peak is selected from 719.7±0.5, 1267.7±0.5, and 1298.0±0.5.

4. The information acquisition method according to claim 3, wherein the m/z of the ion peak is 719.7±0.5.

5. The information acquisition method according to claim 2, wherein the protein is HER2, the digestive enzyme for the limited proteolysis is trypsin, and the ion peak is composed of one or more peaks wherein the m/z of each ion peak is selected from 719.7±0.5, 1267.7±0.5, and 1298.0±0.5.
